(19) United States
(12) Patent Application Publication
(10) Pub. No.: US 2009/0054445 A1
(43) Pub. Date: Feb. 26, 2009

(54) Amide Substituted Quinolines

(75) Inventors: Jeffrey Albert, Wilmington, DE (US); Cristobal Alhambra, Wilmington, DE (US); James Kang, Wilmington, DE (US); Gerard Koether, Wilmington, DE (US); Yan Li, Wilmington, DE (US); Thomas Simpson, Wilmington, DE (US); James Woods, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/162,002

(22) PCT Filed: Jan. 25, 2007
(86) PCT No.: PCT/SE07/00067

§ 371 Date: Jul. 24, 2008

Related U.S. Application Data
(60) Provisional application No. 60/762,762, filed on Jan. 27, 2006.

(51) Int. Cl. A61K 31/4709; C07D 215/20; A61K 31/501; A61P 25/00; C07D 403/12

(52) U.S. Cl. 514/252.04; 546/156; 544/238; 514/312

(57) ABSTRACT

Compounds of Formula I

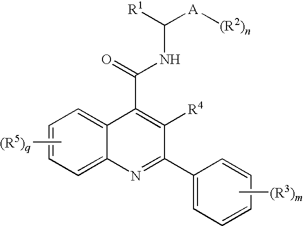

wherein R¹, A, R², n, R³, m, R⁴, R⁵ and q are as described in the specification, pharmaceutically-acceptable salts, methods of making, pharmaceutical compositions containing and methods for using the same.

### FIELD OF THE INVENTION

This invention relates to quinoline derivatives, pharmaceutical compositions comprising them, and the use of such compounds in the treatment of central nervous system and peripheral diseases or disorders. This invention also relates to the use of such compounds in combination with one or more other CNS agents to potentiate the effects of the other CNS agents. The compounds of this invention are also useful as probes for the localization of cell surface receptors.

### BACKGROUND OF THE INVENTION

Tachykinin receptors are the targets of a family of structurally related peptides that include substance P (SP), neurokinin A (NKA) and neurokinin B (NKB), collectively “tachykinins.” Tachykinins are synthesized in the central nervous system (CNS), and peripheral tissues, where they exert a variety of biological activities. Three tachykinin receptors are known which are named neurokinin-1 (NK-1), neurokinin-2 (NK-2) and neurokinin-3 (NK-3) receptors. NK-1 and NK-2 receptors are expressed in a wide variety of peripheral tissues and NK-1 receptors are also expressed in the CNS whereas NK-3 receptors are primarily expressed in the CNS.

The neurokinin receptors mediate a variety of tachykinin-stimulated biological effects that include: transmission of excitatory neuronal signals in the CNS and periphery (e.g. pain signals), modulation of smooth muscle contractile activity, modulation of immune and inflammatory responses, induction of hypotensive effects via dilation of the peripheral vasculature, and stimulation of endocrine and exocrine gland secretions.

In the CNS, activation of NK-3 receptors has been shown to modulate dopamine, acetylcholine and serotonin release, suggesting a therapeutic utility for NK-3 ligands for the treatment of a variety of disorders including anxiety, depression, schizophrenia and obesity. Studies in primate brain have shown the presence of NK-3 mRNA in a variety of regions relevant to these disorders. Studies in rats have shown NK-3 receptors to be located on MCH-containing neurons in the lateral hypothalamus and zona incerta, again suggesting a therapeutic utility for NK-3 ligands for obesity.

Non-peptide ligands have been developed for each of the tachykinin receptors, however known non-peptide NK-3 receptor antagonists suffer from a number of problems such as species selectivity which limits the potential to evaluate these compounds in many appropriate disease models. New non-peptide NK-3 receptor ligands are therefore desirable for use as therapeutic agents and as tools to investigate the biological consequences of NK-3 receptor modulation.

### SUMMARY OF THE INVENTION

Disclosed are compounds, particularly quinoline derivatives with affinity for NK-3 receptors (NK-3r). These compounds have potential for the treatment of a broad array of diseases, disorders and conditions including but not limited to depression, anxiety, schizophrenia, cognitive disorders, psychoses, obesity, inflammatory diseases including irritable bowel syndrome and inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, and testicular cancer in which modulation of the activity of NK-3 receptors is beneficial.

Ligands for NK-3 receptors disclosed and stereoisomers, enantiomers, in vivo-hydrolysable precursors and pharmaceutically-acceptable salts thereof are compounds of Formula I,

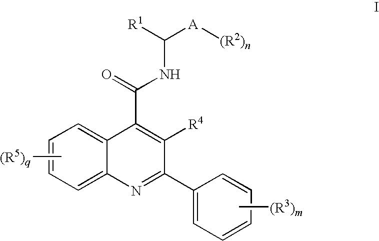

wherein:

R¹ is heterocyclyl or alkyl- or halo-substituted heterocyclyl;

A is phenyl or C₃₋₇cycloalkyl-;

R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;

n is 1, 2 or 3;

R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;

m is 1, 2 or 3;

R⁴ is selected from H, —OH, —NH₂, —OSO₂R⁶, C₁₋₄alkyl-, C₁₋₄alkoxy-, C₁₋₆alkoxyC₁₋₆alkyl-, and E-(CH₂)ₚ—, where E is selected from —CN, —NR⁶R⁷, —SOC₁₋₆alkyl, —SO₂C₁₋₆alkyl, —NR⁶SO₂R⁷, —SR⁶, —N(O⁻)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;

R⁵ at each occurrence is independently selected from H, —OH, —CN, halogen, —R⁶, —OR⁶, —NR⁶R⁷, —SR⁶, —SOR⁶ and —SO₂R⁶;

q is 1, 2 or 3;

wherein:

R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-, and

when R², R³ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen.

Also disclosed are pharmaceutical compositions and formulations containing the compounds, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes. In particular are disclosed compounds, compositions containing them, and methods using them for treating or preventing conditions and disorders associated with a wide range of diseases or disorders in which NK-3 receptors are considered to have a role.

### DETAILED DESCRIPTION OF THE INVENTION

Compounds of the invention are compounds of Formula I,

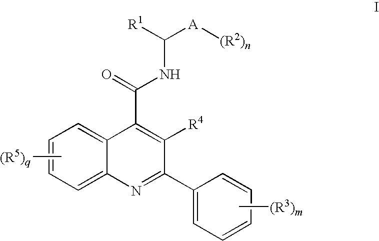

wherein:

R¹ is heterocyclyl or alkyl- or halo-substituted heterocyclyl;

A is phenyl or C₃₋₇cycloalkyl-;

R¹ at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;

n is 1, 2 or 3;

R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;

m is 1, 2 or 3;

R⁴ is selected from H, —OH, —NH₂, —OSO₂R⁶, C₁₋₄alkyl-, C₁₋₄alkoxy-, C₁₋₆alkoxyC₁₋₆alkyl-, and E-(CH₂)ₚ—, where E is selected from —CN, —NR⁶R⁷, —SOC₁₋₆alkyl, —SO₂C₁₋₆alkyl, —NR⁶SO₂R⁷, —SR⁶, —N⁺(O⁻)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;

R⁵ at each occurrence is independently selected from H, —OH, —CN, halogen, —R⁶, —OR⁶, —NR⁶R⁷, —SR⁶, —SOR⁶ and —SO₂R⁶;

q is 1, 2 or 3;

wherein:

R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-, and

when R², R³ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen; stereoisomers, enantiomers, in vivo-hydrolysable precursors and pharmaceutically-acceptable salts thereof.

Particular compounds are those wherein:

A is phenyl;

R² at each occurrence is independently selected from H, halogen, unsubstituted C₁₋₆alkyl and unsubstituted C₁₋₆alkoxy-;

R³ is H or halogen;

R⁴ is OH or an alkyl, cycloalkyl, alkoxy or alkoxyalkyl moiety and said moieties are unsubstituted or have 1, 2, 3, 4 or 5 substituents independently selected at each occurrence from —OH, —NH₂, —CN and halogen;

n and m are both 1 or 2, and

stereoisomers, enantiomers, in vivo-hydrolysable precursors and pharmaceutically-acceptable salts thereof.

Other particular compounds are those wherein:

R¹ is heterocyclyl or methyl- or fluoro-substituted heterocyclyl, stereoisomers, enantiomers, in vivo-hydrolysable precursors and pharmaceutically-acceptable salts thereof.

Yet other particular compounds are those wherein:

R⁵ is H,

stereoisomers, enantiomers, in vivo-hydrolysable precursors and pharmaceutically-acceptable salts thereof.

Other particular compounds are those wherein:

A is phenyl;

R² at each occurrence is independently selected from H, halogen, unsubstituted C₁₋₆alkyl and unsubstituted C₁₋₆alkoxy-;

R³ is H or halogen;

n and m are both 1 or 2;

R⁴ is OH, and

R⁵ is H,

stereoisomers, enantiomers, in vivo-hydrolysable precursors and pharmaceutically-acceptable salts thereof.

Still other particular compounds are those wherein:

R¹ is morpholinyl, furanyl, alkyl-furanyl, tetrahydrofuranyl, benzofuranyl, oxazolyl, dihydro-oxazolyl, isoxyzolyl, 1H-imidazolyl, 1H-alkyl-imidazolyl, pyrazolyl, alkyl-pyrazolyl, oxadiazolyl, alkyl-oxadiazolyl, pyridinyl, pyridazinyl, pyrimidinyl, thiophenyl, thiadiazolyl or thiazolyl;

A is phenyl;

R² at each occurrence is independently selected from H, F, —CH₃ and —OCH₃;

R³ is H or F;

n, m and q are each 1 or 2, and

R⁵ at each occurrence is independently selected from H, —OH and halogen, stereoisomers, enantiomers, in vivo-hydrolysable precursors and pharmaceutically-acceptable salts thereof.

Still other particular compounds are enantiomers in accord with Formula II

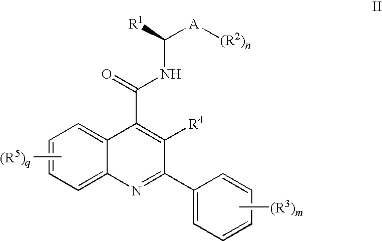

wherein R¹, A, R², n, R³, m, R⁵ and q are as defined for Formula I;
stereoisomers, enantiomers, in vivo-hydrolysable precursors and pharmaceutically-acceptable salts thereof.

Still other particular compounds are those wherein

R¹ is furanyl, oxazolyl, pyridinyl or thiophenyl;

A is phenyl;

R² at each occurrence is independently selected from H, F, —CH₃ and —OCH₃;

R³ is H or F;

n, m and q are each 1 or 2, and

R⁵ at each occurrence is independently selected from H, —OH and halogen, or a stereoisomer, enantiomer, in vivo-hydrolysable precursor or pharmaceutically-acceptable stereoisomers, enantiomers, in vivo-hydrolysable precursors and pharmaceutically-acceptable salts thereof.

Particular compounds are selected from:
3-hydroxy-2-phenyl-quinoline-4-carboxylic acid (furan-2-yl-phenyl-methyl)-amide;
3-hydroxy-N-((1-methyl-1H-pyrazol-5-yl)(phenyl)methyl)-2-phenylquinoline-4-carboxamide;
3-hydroxy-2-phenyl-N-(phenyl(thiophen-2-yl)methyl)quinoline-4-carboxamide;
3-hydroxy-2-phenyl-N-(phenylpyridin-2-yl)methyl)quinoline-4-carboxamide;
2-phenyl-4-{[phenyl(pyridin-2-yl)methyl]carbamoyl} quinolin-3-yl methanesulfonate;
3-[(methylsulfonyl)amino]-2-phenyl-N-[phenyl(pyridin-2-yl)methyl]quinoline-4-carboxamide;
3-[(methylsulfonyl)amino]-2-phenyl-N-[phenyl(2-thienyl)methyl]quinoline-4-carboxamide;
N-[(1-methyl-1H-pyrazol-5-yl)(phenyl)methyl]-3-[(methylsulfonyl)amino]-2-phenylquinoline-4-carboxamide, and
3-[(methylsulfonyl)amino]-N-[1,3-oxazol-5-yl(phenyl)methyl]-2-phenylquinoline-4-carboxamide.

Other particular compounds are selected from:
3-hydroxy-2-phenyl-quinoline-4-carboxylic acid [phenyl-(tetrahydro-furan-2-yl)-methyl]-amide;
3-hydroxy-2-phenyl-quinoline-4-carboxylic acid [(4-methyl-furan-2-yl)-phenyl-methyl]-amide;
3-hydroxy-2-phenyl-quinoline-4-carboxylic acid [(5-methyl-furan-2-yl)-phenyl-methyl]-amide;
3-hydroxy-2-phenyl-quinoline-4-carboxylic acid (benzofuran-2-yl-phenyl-methyl)-amide;
3-hydroxy-2-phenyl-quinoline-4-carboxylic acid (oxazol-2-yl-phenyl-methyl)-amide;
3-hydroxy-2-phenyl-quinoline-4-carboxylic acid [(4,5-dihydro-oxazol-2-yl)-phenyl-methyl]-amide;
3-hydroxy-2-phenyl-quinoline-4-carboxylic acid (isoxazol-5-yl-phenyl-methyl)-amide;
3-hydroxy-2-phenyl-quinoline-4-carboxylic acid [(1H-imidazol-4-yl)-phenyl-methyl]-amide;
3-hydroxy-2-phenyl-quinoline-4-carboxylic acid [(2-methyl-1H-imidazol-4-yl)-phenyl-methyl]-amide;
3-hydroxy-2-phenyl-quinoline-4-carboxylic acid [(1-methyl-1H-imidazol-4-yl)-phenyl-methyl]-amide;
3-hydroxy-2-phenyl-quinoline-4-carboxylic acid [(1-methyl-1H-imidazol-2-yl)-phenyl-methyl]-amide;
3-hydroxy-2-phenyl-quinoline-4-carboxylic acid [(1H-imidazol-2-yl)-phenyl-methyl]-amide;
3-hydroxy-2-phenyl-quinoline-4-carboxylic acid [(1-methyl-1H-pyrazol-3-yl)-phenyl-methyl]-amide;
3-hydroxy-2-phenyl-quinoline-4-carboxylic acid [phenyl-(1H-pyrazol-3-yl)-methyl]-amide;
3-hydroxy-2-phenyl-quinoline-4-carboxylic acid [(3-methyl-[1,2,4]oxadiazol-5-yl)-phenyl-methyl]-amide;
3-hydroxy-2-phenyl-quinoline-4-carboxylic acid [(6-fluoro-pyridin-2-yl)-phenyl-methyl]-amide;
3-hydroxy-2-phenyl-quinoline-4-carboxylic acid (phenyl-pyridazin-3-yl-methyl)-amide;
3-hydroxy-2-phenyl-quinoline-4-carboxylic acid (phenyl-pyrimidin-4-yl-methyl)-amide;
3-hydroxy-2-phenyl-quinoline-4-carboxylic acid [(4,5-dimethyl-furan-2-yl)-phenyl-methyl]-amide;
3-hydroxy-2-phenyl-quinoline-4-carboxylic acid (phenyl-pyrazol-1-yl-methyl)-amide;
3-hydroxy-2-phenyl-quinoline-4-carboxylic acid (phenyl-[1,3,4]thiadiazol-2-yl-methyl)-amide, and
3-hydroxy-2-phenyl-quinoline-4-carboxylic acid [(2-methyl-thiazol-5-yl)-phenyl-methyl]-amide, and stereoisomers, enantiomers, in vivo-hydrolysable precursors and pharmaceutically-acceptable salts thereof.

Compounds of the present invention have the advantage that they may be more soluble, be more easily absorbed and more efficacious in vivo, produce fewer side effects, be less toxic, be more potent, more selective, be longer acting, be less metabolized and/or have a better pharmacokinetic profile than, or have other useful pharmacological or physicochemical properties over known compounds. Using assays for functional activity described herein, compounds of the invention will be found to have IC50's of less than about 1 μM for NK-3 receptors and many compounds will be found to have IC50's of less than about 100 nM for NK-3 receptors.

Compounds of the invention labeled with tritium are useful for the discovery of novel medicinal compounds that bind to and modulate the activity, by agonism, partial agonism, or antagonism, of an NK-3 receptor. Such tritium-labeled compounds may be used in assays that measure the displacement of such compounds to assess the binding of ligands that bind to NK-3 receptors.

In a further aspect the invention relates to compounds described herein additionally comprising one or more atoms of a radioisotope. In a particular form of this aspect of the invention the compound comprises a radioactive halogen. Such radio-labeled compounds are synthesized by incorporating radio-labeled starting materials by known methods. Particular embodiments of this aspect of the invention are those in which the radioisotope is selected from ¹⁸F, ¹²³I, ¹²⁵I, ¹³¹I, ⁷⁵Br, ⁷⁶Br, ⁷⁷Br or ⁸²Br. A most particular embodiment of this aspect of the invention is that in which the radioisotope is ¹⁸F. Such compounds comprising one or more atoms of a radioisotope are useful as positron emission tomography (PET) ligands and for other uses and techniques to determine the location of NK3 receptors.

### Therapeutic Uses of Compounds:

In another aspect the invention relates to compounds in accord with Formula I described herein and the use of such compounds in therapy and in compositions useful for therapy.

In another aspect the invention encompasses the use of compounds described herein for the therapy of diseases mediated through the action of NK-3 receptors. Such an aspect encompasses methods of treatment or prophylaxis of diseases or conditions in which modulation of the NK-3 receptor is beneficial which methods comprise administering a therapeutically-effective amount of an antagonistic compound of the invention to a subject suffering from said disease or condition.

A compound of Formula I or a pharmaceutically acceptable salt, solvate or in vivo hydrolysable ester thereof, or a pharmaceutical composition or formulation comprising a compound of Formula I may be administered concurrently, simultaneously, sequentially or separately with another pharmaceutically active compound or compounds selected from the following:

(i) antidepressants such as amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, robalzotan, sertraline, sibutramine, thionisoxetine, tranylcypromaine, trazodone, trimipramine, venlafaxine and equivalents and pharmaceutically active isomer(s) and metabolite(s) thereof;
(ii) atypical antipsychotics including for example quetiapene and pharmaceutically active isomer(s) and metabolite(s) thereof,
(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutlypiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof;
(iv) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof;
(v) anticonvulsants including for example carbamazepine, valproate, lamotrogine, gabapentin and equivalents and pharmaceutically active isomer(s) and metabolite(s) thereof;
(vi) Alzheimer's therapies including for example donepezil, memantine, tacrine and equivalents and pharmaceutically active isomer(s) and metabolite(s) thereof;
(vii) Parkinson's therapies including for example deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof,
(viii) migraine therapies including for example almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof;
(ix) stroke therapies including for example abciximab, activase, NXY-059, citicoline, crobenetine, desmoteplase, repinotan, traxoprodil and equivalents and pharmaceutically active isomer(s) and metabolite(s) thereof;
(x) urinary incontinence therapies including for example darafenacin, falvoxate, oxybutynin, propiverine, robalzotan, solifenacin, tolterodine and equivalents and pharmaceutically active isomer(s) and metabolite(s) thereof;
(xi) neuropathic pain therapies including for example gabapentin, lidoderm, pregablin and equivalents and pharmaceutically active isomer(s) and metabolite(s) thereof,
(xii) nociceptive pain therapies such as celecoxib, etoricoxib, lumiracoxib, rofecoxib, valdecoxib, diclofenac, loxoprofen, naproxen, paracetamol and equivalents and pharmaceutically active isomer(s) and metabolite(s) thereof;
(xiii) insomnia therapies including for example allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos, secobarbital, zaleplon, zolpidem and equivalents and pharmaceutically active isomer(s) and metabolite(s) thereof, or
(xiv) mood stabilizers including for example carbamazepine, divalproex, gabapentin, lamotrigine, lithium, olanzapine, quetiapene, valproate, valproic acid, verapamil, and equivalents and pharmaceutically active isomer(s) and metabolite(s) thereof.

Such combination products employ the compounds of this invention within the dosage range described herein and the other pharmaceutically active compound or compounds within approved dosage ranges and/or the dosage described in the publication reference.

One embodiment of this aspect of the invention is a method of treatment or prophylaxis of disorders, wherein the disorder is depression, anxiety, schizophrenia, cognitive disorders, psychoses, obesity, inflammatory diseases including irritable bowel syndrome and inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, or testicular cancer comprising administering a pharmacologically effective amount of a compound of Formula I to a patient in need thereof.

A further aspect of the invention is the use of a compound according to the invention, an enantiomer thereof or a pharmaceutically-acceptable salt thereof, in the treatment or prophylaxis of a disease or condition in which modulation of the NK-3 receptor is beneficial. Particular diseases and conditions that may be treated are depression, anxiety, schizophrenia, cognitive disorders, psychoses, obesity, inflammatory diseases including irritable bowel syndrome and inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, and testicular cancer. More particular embodiments encompass uses of a compound in the treatment or prophylaxis of anxiety, depression, schizophrenia and obesity.

A further aspect of the invention is the use of a compound according to the invention, an enantiomer thereof or a pharmaceutically-acceptable salt thereof, in the manufacture of a medicament for the treatment or prophylaxis of the diseases or conditions mentioned herein. A particular embodiment of this aspect of the invention is the use of a compound of the invention in the manufacture of a medicament for treatment or prophylaxis of depression, anxiety, schizophrenia, cognitive disorders, psychoses, obesity, inflammatory diseases including irritable bowel syndrome and inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, and testicular cancer.

### Pharmaceutical Compositions

Compounds of the invention, enantiomers thereof, and pharmaceutically-acceptable salts thereof, may be used on their own or in the form of appropriate medicinal preparations for enteral or parenteral administration. According to a further aspect of the invention, there is provided a pharmaceutical composition including preferably less than 80% and more preferably less than 50% by weight of a compound of the invention in admixture with an inert pharmaceutically-acceptable diluent, lubricant or carrier.

Examples of diluents, lubricants and carriers are

for tablets and dragees: lactose, starch, talc, stearic acid;

for capsules: tartaric acid or lactose;

for injectable solutions: water, alcohols, glycerin, vegetable oils;

for suppositories: natural or hardened oils or waxes.

There is also provided a process for the preparation of such a pharmaceutical composition which process comprises mixing or compounding the ingredients together and forming the mixed ingredients into tablets or suppositories, encapsulating the ingredients in capsules or dissolving the ingredients to form injectable solutions.

Pharmaceutically-acceptable derivatives include solvates and salts. For example, the compounds of the invention may form acid addition salts with acids, such as conventional pharmaceutically-acceptable acids including maleic, hydrochloric, hydrobromic, phosphoric, acetic, fumaric, salicylic, citric, lactic, mandelic, tartaric and methanesulfonic acids.

Acid addition salts of compounds of Formula I may be salts of mineral acids, for example hydrochloride and hydrobromide salts; or salts formed with organic acids such as formate, acetate, maleate, benzoate, tartrate, and fumarate salts. Acid addition salts of compounds of Formula I may be formed by reacting the free base or a salt, enantiomer or protected derivative thereof, with one or more equivalents of an appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

For the uses, methods, medicaments and compositions mentioned herein the amount of compound used and the dosage administered will, of course, vary with the compound employed, the mode of administration and the treatment desired. However, in general, satisfactory results are obtained when the compounds of the invention are administered at a daily dosage of about 0.1 mg to about 20 mg/kg of animal body weight. Such doses may be given in divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carriers, lubricants and diluents.

Some compounds of the invention may exist in tautomeric, enantiomeric, stereoisomeric or geometric isomeric forms, all of which are included within the scope of the invention. Various optical isomers may be isolated by separation of a racemic form of a compound using conventional techniques, e.g. fractional crystallization or chiral HPLC. Alternatively, individual enantiomers may be made by reaction of appropriate optically active starting materials under reaction conditions that will not cause racemization.

### Abbreviations and Definitions

As used herein, unless otherwise indicated, C₁₋₆alkyl includes but is not limited to methyl, ethyl, n-propyl, n-butyl, i-propyl, i-butyl, t-butyl, s-butyl moieties, whether alone or part of another group and alkyl groups may be straight-chained or branched;

As used herein, unless otherwise indicated, C₁₋₆alkoxy includes but is not limited to —O-methyl, —O-ethyl, —O-n-propyl, —O-n-butyl, —O-n-propyl, —O-n-butyl, —O-t-butyl, —O-s-butyl moieties, whether alone or part of another group and alkoxy groups may be straight-chained or branched;

As used herein C₃₋₆cycloalkyl groups include but are not limited to the cyclic alkyl moieties cyclopropyl, cyclobutyl, cyclopentyl and cyclohexyl;

As used herein, unless otherwise indicated, C₂₋₆alkenyl includes but is not limited to 1-propenyl, 2-propenyl, 1-butenyl, 2-butenyl and 3-butenyl;

As used herein, unless otherwise indicated, C₂₋₆alkynyl includes but is not limited to ethynyl, 1-propynyl, 2-propynyl, 1-butynyl, 2-butynyl and 3-butynyl;

As used herein, unless otherwise indicated, halo or halogen refers to fluorine, chlorine, bromine, or iodine;

As used herein, aryl includes to phenyl and naphthyl;

As used herein, heterocyclyl means aromatic or non-aromatic heterocyclic rings and includes but is not limited to unsubstituted or alkyl- or halo-substituted N- or C-linked furyl, imidazolyl, oxazolyl, pyrrolidinyl, thiazolyl, thiophenyl, pyrrolyl, morpholinyl, piperidinyl, piperazinyl, pyrazinyl, pyridyl, pyrimidinyl, indanyl, indolyl, quinolinyl, isoquinolinyl, quinazolinyl, quinoxalinyl, benzo[b]thiophenyl, benzoxazolyl, and benzthiazolyl;

DCM refers to dichloromethane;

EtOAc refers to ethyl acetate;

EDC refers to 1-(3-dimethylaminopropyl)-3-ethylcarbodiimide;

EDTA refers to ethylenediaminetetraacetic acid;

HOBT refers to hydroxybenzotriazole;

HEPES refers to 4-(2-hydroxyethyl)-1-piperazine ethane sulfonic acid, monosodium salt, and

TEA refers to triethylamine.

In processes described herein, where necessary, hydroxy, amino, or other reactive groups may be protected using a protecting group as described in the standard text “Protecting groups in Organic Synthesis”, 3^{rd} Edition (1999) by Greene and Wuts.

Unless otherwise stated, reactions are conducted under an inert atmosphere, preferably under a nitrogen atmosphere and are usually conducted at a pressure of about one to about three atmospheres, preferably at ambient pressure (about one atmosphere).

The compounds of the invention and intermediates may be isolated from their reaction mixtures by standard techniques.

Acid addition salts of compounds of Formula I may be formed by reacting the free base or a salt, enantiomer or protected derivative thereof, with one or more equivalents of an appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin. Acid addition salts of compounds of Formula I which may be mentioned include salts of mineral acids, for example the hydrochloride and hydrobromide salts; and salts formed with organic acids such as formate, acetate, maleate, benzoate, tartrate, and fumarate salts.

### Synthesis and Schemes

Compounds of Formula I may be prepared in general by reacting a 2-phenyl-quinoline-4-carboxylic acid with an activating agent such as thionyl chloride to form the corresponding acid chloride. This can be reacted with a suitable amine in the presence of a base such as triethylamine to afford a 2-phenyl-quinoline-4-carboxylic acid (alkyl)-amide.

An exemplary process, to form a particular compound of Formula I is shown in Scheme 1.

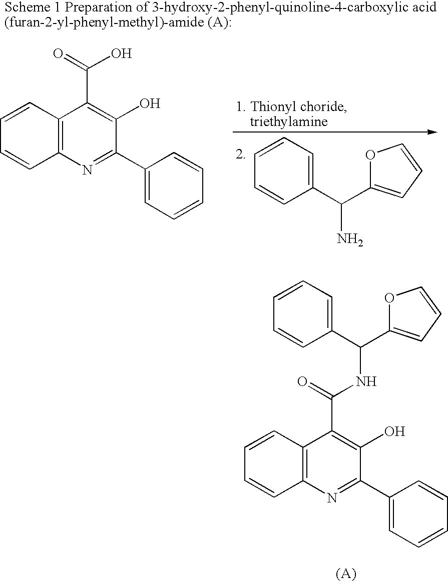

Compounds of the invention may be prepared by processes analogous to that described in Scheme 1. Those of skill in the art will readily appreciate that many suitable amines and acid chlorides and carboxylic acids may be used to form compounds within the scope of the subject matter described herein as Formula I.

The exemplary compounds and processes describe the invention by way of illustration and example for clarity of understanding. However to those skilled in the art, upon contemplation of the teaching of compounds, processes and methods of this invention, modifications and changes will be apparent that may be made thereto without departing from the spirit or scope of the invention.

### EXAMPLE 1

### 3-Hydroxy-2-phenyl-quinoline-4-carboxylic acid (furan-2-yl-phenyl-methyl)-amide

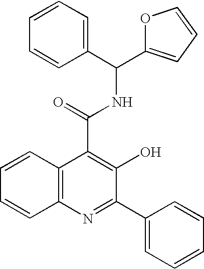

To a solution of 3-hydroxy-2-phenyl-quinoline-4-carboxylic acid (420 mg, 1.58 mmol) and triethylamine (706 uL, 5.1 mmol) in ethyl acetate (10 mL) at 0° C. was added thionyl chloride (111 uL, 1.52 mmol). The ice bath was removed and the solution was allowed to stir at room temperature for 1.5 hr. After this time furan-2-yl-phenyl-methylamine hydrochloride (265 mg, 1.27 mmol) was added, then the temperature was raised to 80° C. After stirring for 1.5 h the mixture was cooled, diluted with methylene chloride, extracted with pH 7 buffer, dried (MgSO₄), filtered, concentrated, and purified by flash silica chromatography with gradient elution using 0% to 10% ethyl acetate in methylene chloride to afford the product as a slightly yellow powder. ¹H NMR (300 MHz, DMSO) δ 9.81 (s, 1H), 9.70 (s, 1H), 7.96 (s, 3H), 7.64-7.32 (m, 12H), 6.51 (d, J=9.7 Hz, 11H), 6.44 (s, 1H), 6.31 (d, J=3.1 Hz, 1H). HRMS m/z 421.1504, calcd for C₂₇H₂₀N₂O₃ 421.1552.

Compounds of Example 2 through 16 may be prepared by a process analogous to that of Example 1.

### EXAMPLE 2

### 3-Hydroxy-2-phenyl-quinoline-4-carboxylic acid [phenyl-(tetrahydro-furan-2-yl)-methyl]-amide

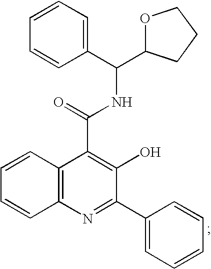

### EXAMPLE 3

### 3-Hydroxy-2-phenyl-quinoline-4-carboxylic acid [(4-methyl-furan-2-yl)-phenyl-methyl]-amide

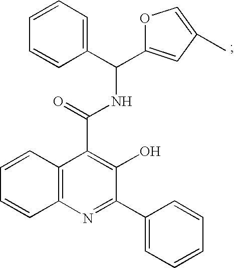

### EXAMPLE 4

### 3-Hydroxy-2-phenyl-quinoline-4-carboxylic acid [(5-methyl-furan-2-yl)-phenyl-methyl]-amide

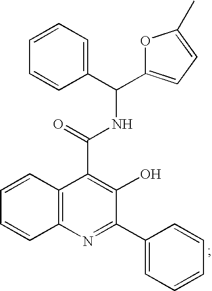

### EXAMPLE 5

### 3-Hydroxy-2-phenyl-quinoline-4-carboxylic acid (benzofuran-2-yl-phenyl-methyl)-amide

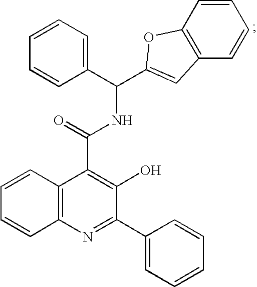

### EXAMPLE 6

### 3-Hydroxy-2-phenyl-quinoline-4-carboxylic acid (oxazol-2-yl-phenyl-methyl)-amide

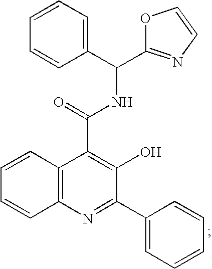

### EXAMPLE 7

### 3-Hydroxy-2-phenyl-quinoline-4-carboxylic acid [(4,5-dihydro-oxazol-2-yl)-phenyl-methyl]-amide

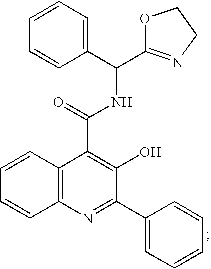

### EXAMPLE 8

### 3-Hydroxy-2-phenyl-quinoline-4-carboxylic acid (isoxazol-5-yl-phenyl-methyl)-amide

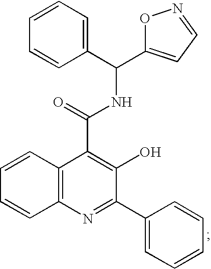

### EXAMPLE 9

### 3-Hydroxy-2-phenyl-quinoline-4-carboxylic acid [(1H-imidazol-4-yl)-phenyl-methyl]-amide

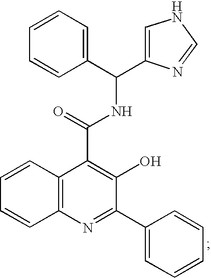

### EXAMPLE 10

### 3-Hydroxy-2-phenyl-quinoline-4-carboxylic acid [(2-methyl-1H-imidazol-4-yl)-phenyl-methyl]-amide

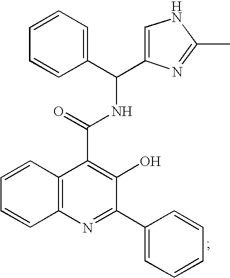

### EXAMPLE 11

### 3-Hydroxy-2-phenyl-quinoline-4-carboxylic acid [(1-methyl-1H-imidazol-4-yl)-phenyl-methyl]-amide

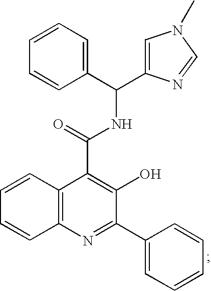

### EXAMPLE 12

### 3-Hydroxy-2-phenyl-quinoline-4-carboxylic acid [(1-methyl-1H-imidazol-2-yl)-phenyl-methyl]-amide

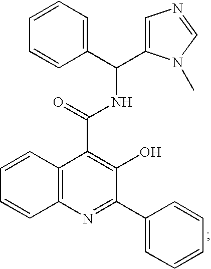

### EXAMPLE 13

### 3-Hydroxy-2-phenyl-quinoline-4-carboxylic acid [(1H-imidazol-2-yl)-phenyl-methyl]-amide

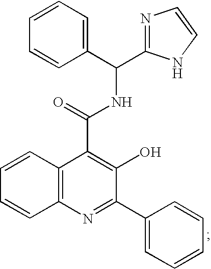

### EXAMPLE 14

### 3-Hydroxy-2-phenyl-quinoline-4-carboxylic acid [(1-methyl-1H-pyrazol-3-yl)-phenyl-methyl]-amide

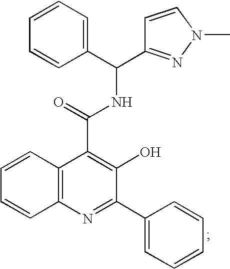

### EXAMPLE 15

### 3-Hydroxy-2-phenyl-quinoline-4-carboxylic acid [phenyl-(1H-pyrazol-3-yl)-methyl]-amide

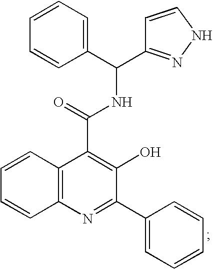

### EXAMPLE 16

### 3-Hydroxy-2-phenyl-quinoline-4-carboxylic acid [(3-methyl-[1,2,4]oxadiazol-5-yl)-phenyl-methyl]-amide

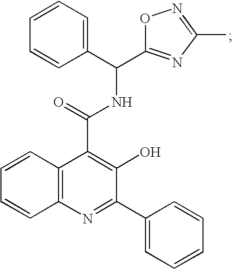

### EXAMPLE 17

### 3-Hydroxy-2-phenyl-N-[phenyl(pyridin-2-yl)methyl]quinoline-4-carboxamide

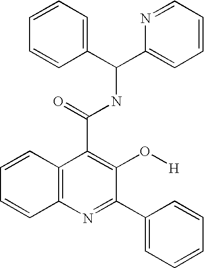

The title compound was prepared using a procedure analogous to that for Example 1 and isolated as a solid. ¹H NMR (300 MHz, CDCl₃) δ 6.53 (d, J=7.2 Hz, 1H), 7.28-7.41 (m, 7H), 7.47-7.60 (m, 5H), 7.71-7.75 (m, 1H), 8.04-8.16 (m, 4H), 8.21-8.25 (m, 1H), 8.63 (d, J=4.8 Hz, 1H), 12.15 (s, 1H). MS ES+, m/z=432 (M+1).

Compounds of Examples 18 through 20 may be prepared by processes analogous to those described herein.

### EXAMPLE 18

### 3-Hydroxy-2-phenyl-quinoline-4-carboxylic acid [(6-fluoro-pyridin-2-yl)-phenyl-methyl]-amide

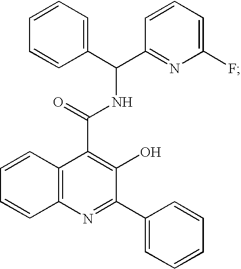

### EXAMPLE 19

### 3-Hydroxy-2-phenyl-quinoline-4-carboxylic acid (phenyl-pyridazin-3-yl-methyl)-amide

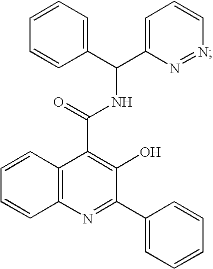

### EXAMPLE 20

### 3-Hydroxy-2-phenyl-quinoline-4-carboxylic acid (phenyl-pyrimidin-4-yl-methyl)-amide

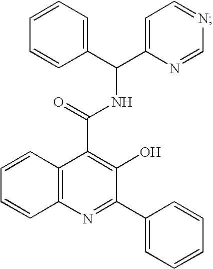

### EXAMPLE 21

### 3-hydroxy-2-phenyl-N-(phenyl(thiophen-2-yl)methyl)quinoline-4-carboxamide

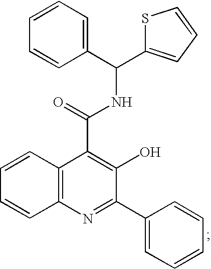

3-hydroxy-2-phenyl-N-(phenyl(thiophen-2-yl)methyl)quinoline-4-carboxamide was prepared using a procedure analogous to that for Example 1. The title compound was isolated as a solid. ¹H NMR (500 MHz, CDCl₃) δ 6.80 (d, J=8.41 Hz, 1H), 6.93 (d, J=3.6 Hz, 1H), 7.0 (dd, J=5.1 Hz, 1H), 7.14 (bd, J=8.41 Hz, 1H), 7.32 (dd, J=5.2 Hz, 1H), 7.41-7.52 (m, 8H), 7.55-7.59 (m, 2H), 8.07 (m, 2H), 8.11-8.16 (m, 2H), 11.16 (s, 1H). MS ES+ m/z 437 (M+1).

Compounds of Examples 22 through 25 may be prepared by processes analogous to those described herein.

### EXAMPLE 22

### 3-Hydroxy-2-phenyl-quinoline-4-carboxylic acid [(4,5-dimethyl-furan-2-yl)-phenyl-methyl]-amide

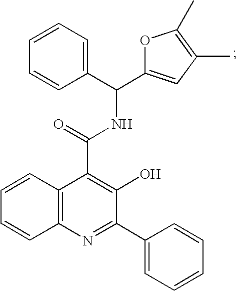

### EXAMPLE 23

### 3-Hydroxy-2-phenyl-quinoline-4-carboxylic acid (phenyl-pyrazol-1-yl-methyl)-amide

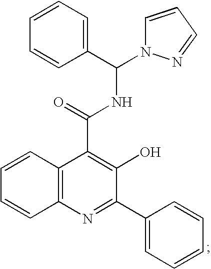

### EXAMPLE 24

### 3-Hydroxy-2-phenyl-quinoline-4-carboxylic acid (phenyl-[1,3,4]thiadiazol-2-yl-methyl)-amide

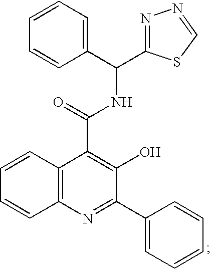

### EXAMPLE 25

### 3-Hydroxy-2-phenyl-quinoline-4-carboxylic acid [(2-methyl-thiazol-5-yl)-phenyl-methyl]-amide

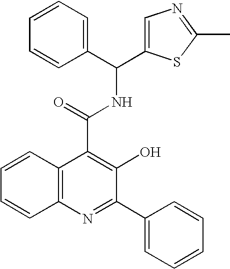

### EXAMPLE 26

### 3-Hydroxy-N-((1-methyl-1H-pyrazol-5-yl)(phenyl)methyl)-2-phenylquinoline-4-carboxamide

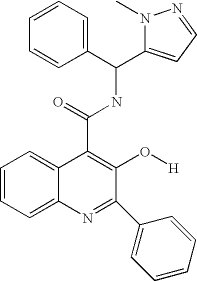

The title compound was prepared using a procedure analogous to that for Example 1 and isolated as a solid. ¹H NMR (500 MHz, CDCl₃) δ 3.83 (s, 3H), 6.20 (d, J=1.71 Hz, 1H), 6.70 (d, J=8.2 Hz, 1H), 7.00 (d, J=8.2 Hz, 1H), 7.35-7.44 (m, 5H), 7.46-7.59 (m, 6H), 7.98 (dd, J=8.0 Hz, 1H), 8.05 (m, 2H), 8.14 (dd, J=8.0 Hz, 1H), 10.8 (s, 1H). MS ES+ m/z=435 (M+1).

### EXAMPLE 27

### 2-phenyl-4-{[phenyl(pyridin-2-yl)methyl]carbamoyl}quinolin-3-yl methanesulfonate

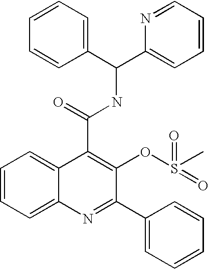

To a solution of 3-hydroxy-2-phenyl-N-[phenyl(pyridin-2-yl)methyl]quinoline-4-carboxamide, Example 17, (29 mg, 0.067 mmol) in dichloromethane (0.5 mL) at room temperature under N₂ triethylamine was added (19 μL, 0.14 mmol). The reaction mixture was cooled to 0° C. and methanesulfonyl chloride (6.4 μL, 0.082 mmol) was added. The reaction mixture was stirred for 15 min at 0° C., then diluted with dichloromethane and washed with an aqueous solution of citric acid (5%), aqueous saturated NaHCO₃ and brine. The organic layer was dried (Na₂SO₄), filtered and concentrated to provide the title compound as a solid. ¹H NMR (300 MHz, CDCl₃) δ 2.46 (s, 3H), 6.44 (d, J=6.6 Hz, 1H), 7.19-7.32 (m, 3H), 7.36-7.40 (m, 2H), 7.48-7.55 (m, 5H), 7.64-7.78 (m, 2H), 7.84-7.91 (m, 3H), 8.18 (d, J=8.5 Hz, 1H), 8.55 (m, 2H). MS ES+, m/z=510 (M+1).

### EXAMPLE 28

### 3-[(methylsulfonyl)amino]-2-phenyl-N-[phenyl(pyridin-2-yl)methyl]quinoline-4-carboxamide

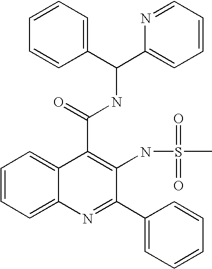

To 3-[(methylsulfonyl)amino]-2-phenylquinoline-4-carboxylic acid (150 mg, 0.44 mmol) HOBT hydrate (89.0 mg, 0.66 mmol) and EDC (125 mg, 0.66 mmol) were added and the mixture was diluted with dichloromethane (3 mL). 4-methylmorpholine (218 μL, 2.0 mmol) was then added and the reaction mixture was stirred at RT under N₂ for 5 minutes before the addition of phenyl(-2-pyridyl)methylamine dihydrochloride (113 mg, 0.44 mmol). The reaction mixture was stirred overnight at RT, diluted with dichloromethane and washed with 10% aqueous citric acid, saturated NaHCO₃ and brine. The organic layer was dried (Na₂SO₄), filtered and concentrated. The title compound was cleanly isolated after etheral trituration. ¹H NMR (500 MHz, CDCl₃) δ 2.30 (s, 3H), 6.54 (d, J=7.2 Hz, 1H), 7.24-7.27 (m, 2H), 7.32 (m, 1H), 7.37 (m, 2H), 7.43-7.46 (m, 3H), 7.51 (m, 2H), 7.55 (m, 1H), 7.68 (m, 1H), 7.74 (m, 1H) 7.86 (d, J=7.4 Hz, 2H), 7.94 (d, J=8.2 Hz, 1H), 7.99 (d, J=7.2 Hz, 1H), 8.16 (d, J=8.5 Hz, 1H), 8.66 (d, J=5.03 Hz, 1H). MS ES+, m/z=509 (M+1).

The starting acid, 3-[(methylsulfonyl)amino]-2-phenylquinoline-4-carboxylic acid was prepared in the following manner:

### a) N-(2-oxo-2-phenylethyl)methanesulfonamide

To a solution of 2-amino-1-phenylethanonehydrochloride (1715 mg, 10 mmol) in DCM (20 mL) was added TEA (2.8 mL, 20 mmol). Upon cooling under ice-water bath, methylsulfonyl chloride (0.77 mL, 10 mmol) was added slowly and the reaction mixture stirred at RT for 12 h. The mixture was partitioned between dichloromethane and brine, dried over sodium sulfate and then concentrated in vacuo to give the title compound as an off white solid. MS APCI, m/z=214 (M+1). LCMS: 1.19 min.

### b) 3-[(methylsulfonyl)amino]-2-phenylquinoline-4-carboxylic acid

To isatin (441 mg, 3 mmol) was added a solution of sodium hydroxide (1.15 g, 29.0 mmol) in water (2.5 mL). The resulting brown precipitate was stirred vigorously at RT for 20 minutes before being heated to 85° C. A solution of N-(2-oxo-2-phenylethyl)methanesulfonamide (639 mg, 3.0 mmol) in ethanol/THF/water (6.3 mL/1.25 ml/6.3 mL was then added dropwise over 30 minutes. The reaction mixture was stirred at 85° C. for further 4 h before cooling to rt. All organic solvents were removed in vacuo and the aqueous residue reduced to a volume of approximately 6 mL. The aqueous residue was washed with ether (3×10 mL) and then the aqueous phase was acidified with cooling to pH 4 with concentrated HCl. The precipitate formed were collected, washed with water and dried to give the title compound as a solid. ¹H NMR (300 MHz, CDCl₃) δ 3.11 (s, 3H), 7.05 (d, 1H), 7.39 (d, 2H), 7.64 (m, 2H), 7.78 (m, 1H), 8.06 (m, 1H), 8.19 (m, 1H), 8.47 (m, 1H), 10.03 (b, 2H). MS APCI, m/z=343 (M+1). LCMS: 1.07 min.

### EXAMPLE 29

### 3-[(methylsulfonyl)amino]-2-phenyl-N-[phenyl(2-thienyl)methyl]quinoline-4-carboxamide

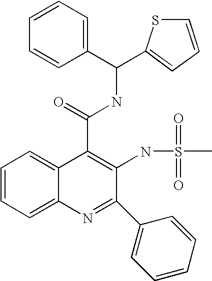

The title compound was prepared using a procedure analogous to that for Example 28 and isolated as a solid after column chromatography using a gradient of 0-60% ethyl acetate/hexanes. ¹H NMR (500 MHz, CDCl₃) δ 2.21 (s, 3H), 6.77 (d, J=7.6 Hz, 1H), 6.97-6.99 (m, 1H), 7.05 (d, J=3.3 Hz, 1H), 7.26-7.30 (m, 2H), 7.33-7.36 (m, 1H), 7.41 (m, 2H), 7.45-7.52 (m, 5H) 7.55-7.58 (m, 1H), 7.73-7.77 (m, 3H), 7.85 (d, J=8.4 Hz, 1H), 8.15 (d, J=8.2 Hz, 1H). MS ES+, m/z=514 (M+1).

### EXAMPLE 30

### N-[(1-methyl-1H-pyrazol-5-yl)(phenyl)methyl]-3-[(methylsulfonyl)amino]-2-phenylquinoline-4-carboxamide

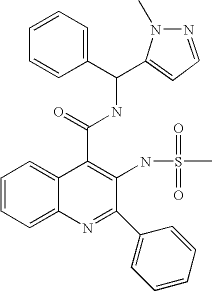

The title compound was prepared using a procedure analogous to that for Example 28 and isolated as a solid after column chromatography using a gradient of 0-60% ethyl acetate/hexanes. ¹H NMR (500 MHz, CDCl₃) δ 2.17 (s, 3H), 3.82 (s, 3H), 6.33 (d, J=1.9 Hz, 1H), 6.6 (d, J=8.0 Hz, 1H) 7.32-7.41 (m, 6H), 7.45-7.52 (m, 4H), 7.56-7.59 (m, 1H), 7.71-7.73 (m, 2H), 7.76-7.78 (m, 2H), 8.16 (d, J=8.1 Hz, 1H). MS ES+, m/z=512 (M+1).

### EXAMPLE 31

### 3-[(methylsulfonyl)amino]-N-[1,3-oxazol-5-yl(phenyl)methyl]-2-phenylquinoline-4-carboxamide

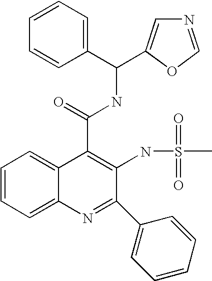

The title compound was prepared using a procedure analogous to that for Example 28. Column chromatography, using a gradient of 0-60% ethyl acetate/hexanes, was used to separate the title compound from material retaining the triisopropylsilyl substituent on the oxazole ring. ¹H NMR (500 MHz, CDCl₃) δ 2.18 (s, 3H), 6.99 (d, J=7.9 Hz, 1H), 7.07 (s, 1H), 7.35-7.43 (m, 3H), 7.45-7.53 (m, 6H), 7.59 (m, 1H), 7.73-7.75 (m, 2H), 7.76-7.78 (m, 1H) 7.82 (d, J=8.6 Hz, 1H), 7.87 (s, 1H), 8.16 (d, J=8.2 Hz, 1H). MS ES+ m/z=499 (M+1).

The precursor amine, 1-phenyl-1-[2-(triisopropylsilyl)-1,3-oxazol-5-yl]methenamine, was prepared in the following manner:

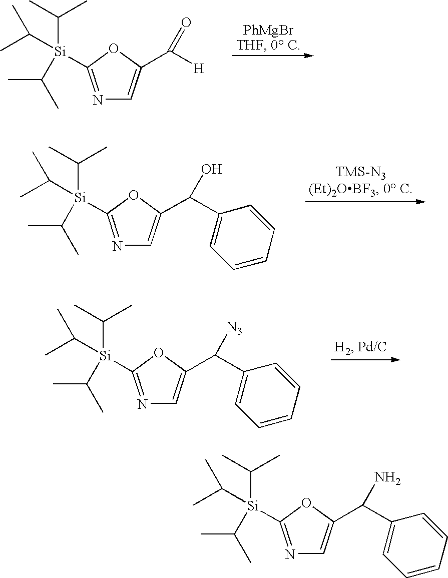

### a) phenyl[2-(triisopropylsilyl)-1,3-oxazol-5-yl]methanol

To a solution of 2-triisopropylsilyl-oxazole-5-carboxaldehyde (1.1 g, 4.3 mmol) in THF (15 mL) at 0° C. under N₂ was added phenylmagnesium bromide (3.0 M etheral solution, 3 mL, 8.8 mmol) over a ten minute period. The reaction mixture was stirred at 0° C. for two hours, then allowed to gradually warm to RT with stirring overnight. The reaction mixture was poured into a flask charged with ice, saturated NH₄Cl (25 mL) and dichloromethane (25 mL). The biphasic mixture was stirred for 30 minutes, then the organic layer was isolated and the aqueous extracted once with dichloromethane. The combined dichloromethane layer was washed with brine, dried (Na₂SO₄), filtered and concentrated. The product was purified via column chromatography using a gradient of 0-40% ethyl acetate/hexane as the eluent. ¹H NMR (500 MHz, CDCl₃) δ 1.1 (d, J=7.2 Hz, 18H), 1.38 (m, 3H), 2.38 (d, J=4.8 Hz, 1H), 5.92 (d, J=4.8 Hz, 1H), 6.92 (s, 1H), 7.31-7.38 (m, 3H), 7.42-7.43 (m, 2H). MS ES+ m/z=332 (M+1).

### b) 5-[azido(phenyl)methyl]-2-(triisopropylsilyl)-1,3-oxazole

To a solution of phenyl[2-(triisopropylsilyl)-1,3-oxazol-5-yl]methanol (331 mg, 1.0 mmol) in toluene (2.0 mL) was added dropwise azidotrimethylsilane (0.66 mL, 5.0 mmol) with stirring. The reaction mixture was cooled to 0° C. and boron trifluoride etherate was added dropwise and the solution was stirred at 0° C. for 2 hours and then at RT overnight. The reaction mixture was carefully diluted with ethyl acetate (5 mL) and saturated NaHCO₃ (10 mL) and stirred for several hours. The organic layer was isolated and the aqueous was extracted with ethyl acetate. The combined ethyl acetate layer was washed with brine, dried (Na₂SO₄), filtered and concentrated. The product was used without further purification. MS ES+ m/z=357 (M+1).

### c) 1-phenyl-1-[2-(triisopropylsilyl)-1,3-oxazol-5-yl]methenamine

A mixture of 5-[azido(phenyl)methyl]-2-(triisopropylsilyl)-1,3-oxazole and 10% Pd/C in EtOH (7 mL) was hydrogenated at 45 psi at RT overnight. The catalyst was filtered through a bed of diatomaceous earth, the diatomaceous earth was thoroughly washed with EtOH, and the filtrate was concentrated via rotary evaporator. The product was used without further purification. MS ES+ m/z=332 (M+1).

### Biological Tests

NK-3 Receptor Binding Activity:

Generally, NK-3r binding activity may be assessed using assays performed as described in Krause et al., (Proc. Natl. Acad. Sci. USA 94: 310-315, 1997). NK-3r complementary DNA is cloned from human hypothalamic RNA using standard procedures. The receptor cDNA is inserted into a suitable expression vector transfected into a Chinese hamster ovary cell line, and a stably-expressing clonal cell line may be isolated, characterized and used for experiments.

Cells may be grown in tissue culture medium by techniques known to those of skill in the art and recovered by low speed centrifugation. Cell pellets may be homogenized, total cellular membranes isolated by high speed centrifugation and suspended in buffered saline. Generally, receptor binding assays may be performed by incubating suitable amounts of purified membrane preparations with ¹²⁵I-methylPhe7-neurokinin B, in the presence or absence of test compounds. Membrane proteins may be harvested by rapid filtration and radioactivity may be quantitated in a P-plate scintillation counter. Nonspecific binding may be distinguished from specific binding by use of suitable controls and the affinity of compounds for the expressed receptor may be determined by using different concentrations of compounds.

Preparation of Membranes from CHO Cells Transfected with Cloned NK-3 Receptors:

A human NK-3 receptor gene was cloned using methods similar to those described for other human NK receptors (Aharony et al., Mol. Pharmacol. 45:9-19, 1994; Caccese et al., Neuropeptides 33, 239-243, 1999). The DNA sequence of the cloned NK-3 receptor differed from the published sequence (Buell et al., FEBS Letts. 299, 90-95, 1992; Huang et al., Biochem. Biophys. Res. Commun. 184, 966-972, 1992) having a silent single T>C base change at nucleotide 1320 of the coding sequence. Since the change is silent, the cloned gene provides a primary amino acid sequence for the encoded NK-3 receptor protein identical to the published sequence. The receptor cDNA was used to transfect CHO-K1 cells using standard methods and a clone stably-expressing the receptor was isolated and characterized. Plasma membranes from these cells were prepared as published (Aharony et al., 1994).

Cells were harvested and centrifuged to remove medium. The pelleted cells were homogenized (Brinkman Polytron, three 15 sec bursts on ice) in a buffer consisting of 50 mM Tris-HCl (pH 7.4), 120 mM NaCl, 5 mM KCl, 10 mM EDTA and protease inhibitors (0.1 mg/ml soybean trypsin inhibitor, and 1 mM iodoacetamide). The homogenate was centrifuged at 1000×g for 10 min at 4° C. to remove cell debris. Pellets were washed once with homogenizing buffer. Supernatants were combined and centrifuged at 40,000×g for 20 min at 4° C. The membrane-containing pellet was homogenized with a Polytron as before. The suspension was centrifuged at 40,000×g for 20 min at 4° C., the pellet suspended in buffer (20 mM HEPES, pH 7.4 containing 3 mM MgCl₂, 30 mM KCl, and 100 μM thiorphan) and the protein concentration determined. The membrane suspension was then diluted to 3 mg/ml with buffer containing 0.02% BSA, and flash frozen. Samples were stored at −80° C. until used.

### Assay for NK-3 Receptor Binding Activity:

A receptor binding assay method with [¹²⁵I]-MePhe7-NKB was modified from that described by Aharony et al., J. Pharmacol. Exper. Ther., 274:1216-1221, 1995.

Competition experiments were carried out in 0.2 mL assay buffer (50 mM Tris-HCl, 4 mM MnCl₂, 10 μM thiorphan, pH 7.4) containing membranes (2 μg protein/reaction), tested competitors, and [¹²⁵I]-MePhe7NKB (0.2 nM). Unlabeled homologue ligand (0.5 μM) was used to define nonspecific binding. Incubations were carried out at 25° C. for 90 min. Receptor-bound ligand was isolated by vacuum filtration in a Packard Harvester onto GF/C plates presoaked in 0.5% BSA. Plates were washed with 0.02 M Tris, pH 7.4. Computation of equilibrium binding constants (K_{D} and Ki), receptor density (Bmax), and statistical analysis was carried out as published previously (Aharony et al., 1995) using GraphPad Prism or IDBS XLfit software.

### NK-3 Functional Activity:

Generally, NK-3 functional activity may be assessed by using calcium mobilization assays in stable NK-3r-expressing cell lines. Calcium mobilization induced by the methylPhe7-neurokinin B agonist may be monitored using a FLIPR (Molecular Devices) instrument in the manner described by the manufacturer. Agonists may be added to the cells and fluorescence responses continuously recorded for up to 5 min. The actions of antagonists may be assessed by preincubating cells prior to administration of the methylPhe7-neurokinin B agonist. The action of agonists may be assessed by observing their intrinsic activity in such a system.

### Assay for NK-3 Functional Activity:

NK-3 receptor expressing CHO cells were maintained in growth media (Ham's F12 medium, 10% FBS, 2 mM L-glutamine, and 50 mg/mL Hygromycin B). One day prior to the assay cells were dispensed into 384-well plates in Ultraculture media (Cambrex Bio Science) with 2 mM L-glutamine to achieve 70-90% confluency. To quantify NK-3 receptor-induced calcium mobilization, cells were first washed with assay buffer consisting of Hanks' Balanced Salt Solution, 15 mM HEPES, and 2.5 mM probenecid, pH 7.4. The cells were then loaded with Fluo4/AM dye (4.4 μM) in assay buffer. Cells were incubated for one hour and then washed with assay buffer, exposed to 0.02-300 nM senktide and the fluorescence response recorded using a FLIPR instrument Molecular Devices Corporation). To quantify antagonism of the agonist response, cells were preincubated with varying concentrations of test compound for 2-20 min and then exposed to 2 μM senktide, a concentration that alone elicits about an 70% maximal calcium response. The resulting data was analyzed using XLfit software (IDBS manufacturer) to determine EC50 and IC50 values.

1. A compound in accord with Formula I 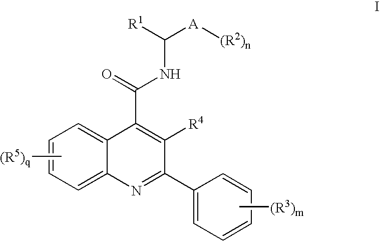
wherein:
R¹ is heterocyclyl or alkyl- or halo-substituted heterocyclyl;
A is phenyl or C₃₋₇cycloalkyl-;
R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
n is 1, 2 or 3;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
R⁴ is selected from H, —OH, —NH₂, —OSO₂R⁶, C₁₋₄alkyl-, C₁₋₄alkoxy-, C₁₋₆alkoxyC₁₋₆alkyl-, and E-(CH₂)ₚ—, where E is selected from —CN, —NR⁶R⁷, —SOC₁₋₆alkyl, —SO₂C₁₋₆alkyl, —NR⁶SO₂R⁷, —SR⁶, —N(O)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇-carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-, and
when R², R³ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen;
or a stereoisomer, enantiomer, in vivo-hydrolysable precursor or pharmaceutically-acceptable salt thereof.

2. A compound according to claim 1, wherein:
A is phenyl;
R² at each occurrence is independently selected from H, halogen, unsubstituted C₁₋₆alkyl and unsubstituted C₁₋₆alkoxy-;
R³ is H or halogen, and
n and m are both 1 or 2;
or a stereoisomer, enantiomer, in vivo-hydrolysable precursor or pharmaceutically-acceptable salt thereof.

3. A compound according to claim 2, wherein:
R⁵ is H,
or a stereoisomer, enantiomer, in vivo-hydrolysable precursor or pharmaceutically-acceptable salt thereof.

4. A compound according to claim 1, wherein R¹ is heterocyclyl or methyl- or fluoro-substituted heterocyclyl,
or a stereoisomer, enantiomer, in vivo-hydrolysable precursor or pharmaceutically-acceptable salt thereof.

5. A compound according to claim 1, wherein:
A is phenyl;
R² at each occurrence is independently selected from H, halogen, unsubstituted C₁₋₆alkyl and unsubstituted C₁₋₆alkoxy-;
R³ is H or halogen;
n and m are both 1 or 2;
R⁴ is OH, and
R⁵ is H,
stereoisomers, enantiomers, in vivo-hydrolysable precursors and pharmaceutically-acceptable salts thereof.

6. A compound according to claim 1, wherein:
R¹ is morpholinyl, furanyl, alkyl-furanyl, tetrahydrofuranyl, benzofuranyl, oxazolyl, dihydro-oxazolyl, isoxyzolyl, 1H-imidazolyl, 1H-alkyl-imidazolyl, pyrazolyl, alkyl-pyrazolyl, oxadiazolyl, alkyl-oxadiazolyl, pyridinyl, pyridazinyl, pyrimidinyl, thiophenyl, thiadiazolyl or thiazolyl;
A is phenyl;
R² at each occurrence is independently selected from H, F, —CH₃ and —OCH₃;
R³ is H or F;
n, m and q are each 1 or 2, and
R⁵ at each occurrence is independently selected from H, —OH and halogen,
or a stereoisomer, enantiomer, in vivo-hydrolysable precursor or pharmaceutically-acceptable salt thereof.

7. A compound according to claim 1, in accord with Formula II 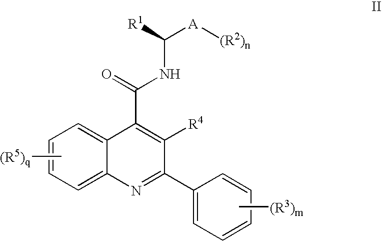
wherein R¹, A, R², n, R³, m, R⁵ and q are as defined for Formula I;
or a stereoisomer, enantiomer, in vivo-hydrolysable precursor or pharmaceutically-acceptable salt thereof.

8. A compound according to claim 7, wherein:
R¹ is furanyl, oxazolyl, pyridinyl or thiophenyl;
A is phenyl;
R² at each occurrence is independently selected from H, F, —CH₃ and —OCH₃;
R³ is H or F;
n, m and q are each 1 or 2, and
R⁵ at each occurrence is independently selected from H, —OH and halogen,
or a stereoisomer, enantiomer, in vivo-hydrolysable precursor or pharmaceutically-acceptable salt thereof.

9. A compound according to claim 1, selected from:
3-hydroxy-2-phenyl-quinoline-4-carboxylic acid (furan-2-yl-phenyl-methyl)-amide;
3-hydroxy-N-((1-methyl-1H-pyrazol-5-yl)(phenyl)methyl)-2-phenylquinoline-4-carboxamide;
3-hydroxy-2-phenyl-N-(phenyl(thiophen-2-yl)methyl)quinoline-4-carboxamide;
3-hydroxy-2-phenyl-N-(phenyl(pyridin-2-yl)methyl)quinoline-4-carboxamide;
2-phenyl-4-{[phenyl(pyridin-2-yl)methyl]carbamoyl} quinolin-3-yl methanesulfonate;
3-[(methylsulfonyl)amino]-2-phenyl-N-[phenyl(pyridin-2-yl)methyl]quinoline-4-carboxamide;
3-[(methyl sulfonyl)amino]-2-phenyl-N-[phenyl(2-thienyl)methyl]quinoline-4-carboxamide;
N-[(1-methyl-1H-pyrazol-5-yl)(phenyl)methyl]-3-[(methylsulfonyl)amino]-2-phenylquinoline-4-carboxamide, and
3-[(methylsulfonyl)amino]-N-[1,3-oxazol-5-yl(phenyl)methyl]-2-phenylquinoline-4-carboxamide,
or a stereoisomer, enantiomer, in vivo-hydrolysable precursor or pharmaceutically-acceptable salt thereof.

10. A compound according to claim 1 selected from:
3-hydroxy-2-phenyl-quinoline-4-carboxylic acid [phenyl-(tetrahydro-furan-2-yl)-methyl]-amide;
3-hydroxy-2-phenyl-quinoline-4-carboxylic acid [(4-methyl-furan-2-yl)-phenyl-methyl]-amide;
3-hydroxy-2-phenyl-quinoline-4-carboxylic acid [(5-methyl-furan-2-yl)-phenyl-methyl]-amide;
3-hydroxy-2-phenyl-quinoline-4-carboxylic acid (benzofuran-2-yl-phenyl-methyl)-amide;
3-hydroxy-2-phenyl-quinoline-4-carboxylic acid (oxazol-2-yl-phenyl-methyl)-amide;
3-hydroxy-2-phenyl-quinoline-4-carboxylic acid [(4,5-dihydro-oxazol-2-yl)-phenyl-methyl]-amide;
3-hydroxy-2-phenyl-quinoline-4-carboxylic acid (isoxazol-5-yl-phenyl-methyl)-amide;
3-hydroxy-2-phenyl-quinoline-4-carboxylic acid [(1H-imidazol-4-yl)-phenyl-methyl]-amide;
3-hydroxy-2-phenyl-quinoline-4-carboxylic acid [(2-methyl-1H-imidazol-4-yl)-phenyl-methyl]-amide;
3-hydroxy-2-phenyl-quinoline-4-carboxylic acid [(1-methyl-1H-imidazol-4-yl)-phenyl-methyl]-amide;
3-hydroxy-2-phenyl-quinoline-4-carboxylic acid [(1-methyl-1H-imidazol-2-yl)-phenyl-methyl]-amide;
3-hydroxy-2-phenyl-quinoline-4-carboxylic acid [(1H-imidazol-2-yl)-phenyl-methyl]-amide;
3-hydroxy-2-phenyl-quinoline-4-carboxylic acid [(1-methyl-1H-pyrazol-3-yl)-phenyl-methyl]-amide;
3-hydroxy-2-phenyl-quinoline-4-carboxylic acid [phenyl-(1H-pyrazol-3-yl)-methyl]-amide;
3-hydroxy-2-phenyl-quinoline-4-carboxylic acid [(3-methyl-[1,2,4]oxadiazol-5-yl)-phenyl-methyl]-amide;
3-hydroxy-2-phenyl-quinoline-4-carboxylic acid [(6-fluoro-pyridin-2-yl)-phenyl-methyl]-amide;
3-hydroxy-2-phenyl-quinoline-4-carboxylic acid (phenyl-pyridazin-3-yl-methyl)-amide;
3-hydroxy-2-phenyl-quinoline-4-carboxylic acid (phenyl-pyrimidin-4-yl-methyl)-amide;
3-hydroxy-2-phenyl-quinoline-4-carboxylic acid [(4,5-dimethyl-furan-2-yl)-phenyl-methyl]-amide;
3-hydroxy-2-phenyl-quinoline-4-carboxylic acid (phenyl-pyrazol-1-yl-methyl)-amide;
3-hydroxy-2-phenyl-quinoline-4-carboxylic acid (phenyl-[1,3,4]thiadiazol-2-yl-methyl)-amide, and
3-hydroxy-2-phenyl-quinoline-4-carboxylic acid [(2-methyl-thiazol-5-yl)-phenyl-methyl]-amide,
or a stereoisomer, enantiomer, in vivo-hydrolysable precursor or pharmaceutically-acceptable salt thereof.

11. A process for preparing a compound of Formula I, 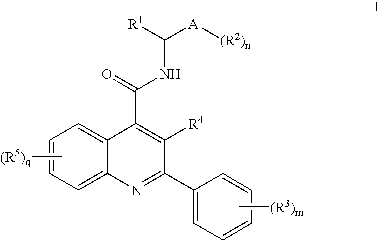
wherein:
R¹ is heterocyclyl or alkyl- or halo-substituted heterocyclyl;
A is phenyl or C₃₋₇cycloalkyl-;
R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
n is 1, 2 or 3;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
R⁴ is selected from H, —OH, —NH₂, —OSO₂R⁶, C₁₋₄alkyl-, C₁₋₄alkoxy-, C₁₋₆alkoxyC₁₋₆alkyl-, and E-(CH₂)ₚ—, where E is selected from —CN, —NR⁶R⁷, —SOC₁₋₆alkyl, —SO₂C₁₋₆alkyl, —NR⁶SO₂R⁷, —SR⁶, —N⁺(O⁻)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-, and
when R², R³ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen;
or a stereoisomer, enantiomer, in vivo-hydrolysable precursor or pharmaceutically-acceptable salt thereof.
said process comprising:
reacting a 2-phenyl-quinoline-4-carboxylic acid with an amine in the presence of a dehydrating agent, to afford a 2-phenyl-quinoline-4-carboxylic acid (alkyl)-amide;
or
reacting toluene-4-sulfonic acid 2-tert-butoxycarbonylamino-2-phenyl-ethyl ester with sodium methanethiolate to afford a (2-methylsulfanyl-1-phenyl-ethyl)-carbamic acid tert-butyl ester;
reacting said butyl ester with trifluoroacetic acid in methylene chloride to afford a 2-methylsulfanyl-1-phenyl-ethyl amine,
reacting said amine with a substituted 2-phenyl-quinoline-4-carboxylic acid in the presence of a dehydrating agent, to afford a compound of Formula I.

12. A method of treatment or prophylaxis of a disease or condition in which modulation of the NK-3 receptor is beneficial which method comprises administering to a subject suffering from said disease or condition a therapeutically-effective amount of a compound in accord with Formula I: 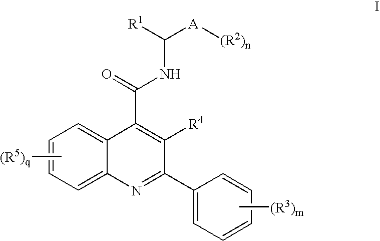
wherein:
R¹ is heterocyclyl or alkyl- or halo-substituted heterocyclyl;
A is phenyl or C₃₋₇cycloalkyl-;
R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
n is 1, 2 or 3;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
R⁴ is selected from H, —OH, —NH₂, —OSO₂R⁶, C₁₋₄alkyl-, C₁₋₄alkoxy-, C₁₋₆alkoxyC₁₋₆alkyl-, and E-(CH₂)ₚ—, where E is selected from —CN, —NR⁶R⁷, —SOC₁₋₆alkyl, —SO₂C₁₋₆alkyl, —NR⁶SO₂R⁷, —SR⁶, —N⁺(O⁻)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-, and
when R², R³ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen;
or a stereoisomer, enantiomer, in vivo-hydrolysable precursor or pharmaceutically-acceptable salt thereof.

13. The method of claim 12, wherein said disease or condition is selected from depression, anxiety, schizophrenia, cognitive disorders, psychoses, obesity, inflammatory diseases, irritable bowel syndrome, inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, and testicular cancer.

14. A pharmaceutical composition comprising a pharmaceutically-acceptable diluent, lubricant or carrier and a compound in accord with Formula I: 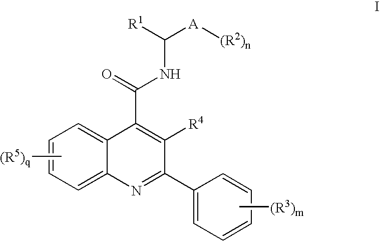
wherein:
R¹ is heterocyclyl or alkyl- or halo-substituted heterocyclyl;
A is phenyl or C₃₋₇cycloalkyl-;
R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
n is 1, 2 or 3;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
R⁴ is selected from H, —OH, —NH₂, —OSO₂R⁶, C₁₋₄alkyl-, C₁₋₄alkoxy-, C₁₋₆alkoxyC₁₋₆alkyl-, and E-(CH₂)ₚ—, where E is selected from —CN, —NR⁶R⁷, —SOC₁₋₆alkyl, —SO₂C₁₋₆alkyl, —NR⁶SO₂R⁷, —SR⁶, —N⁺(O⁻)R⁶R⁷, aryl and an N- or C-linked 5- or 6-membered aromatic or non-aromatic heterocyclic ring having 1, 2, 3 or 4 nitrogen atoms or an N-oxide thereof, and p is 0, 1, 2, 3, 4 or 5;
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-, and
when R², R³ or R⁴ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen;
or a stereoisomer, enantiomer, in vivo-hydrolysable precursor or pharmaceutically-acceptable salt thereof.

15. A method of treatment or prophylaxis of a disease or condition in which modulation of the NK-3 receptor is beneficial which method comprises administering a therapeutically-effective amount of a pharmaceutical composition according to claim 14 to a subject suffering from said disease or condition.

16. The method of claim 15, wherein said disease or condition is selected from depression, anxiety, schizophrenia, cognitive disorders, psychoses, obesity, inflammatory diseases, irritable bowel syndrome, inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, and testicular cancer.

17-20. (canceled)
